(19) United States
(12) Patent Application Publication
(10) Pub. No.: US 2023/0138130 A1
(43) Pub. Date: May 4, 2023

(54) NOVEL COMPOUNDS FOR PREPARATION OF MICROTUBULE ASSOCIATED TAU PROTEIN IMAGING AGENTS, PREPARATION METHODS AND MEDICINAL USES THEREOF

(71) Applicant: Institute of Nuclear Energy Research Atomic Energy Council, R.O.C., Taoyuan (TW)

(72) Inventors: Ming-Hsin Li, Taoyuan (TW); Shih-Wei Lo, Taoyuan (TW); Kai-Hung Cheng, Taoyuan (TW); Liang-Hsun Huang, Taoyuan (TW); Li-Ming Wang, Taoyuan (TW)

(21) Appl. No.: 17/580,884

(22) Filed: Jan. 21, 2022

(30) Foreign Application Priority Data
Oct. 28, 2021 (TW) 110140012

(51) Int. Cl. C07D 471/04

(52) U.S. Cl. CPC C07D 471/04

(57) ABSTRACT

The present disclosure provides a compound shown as formula (I), formula (II), which is used to prepare a radiocontrast tracer that can target binding to Tau protein.

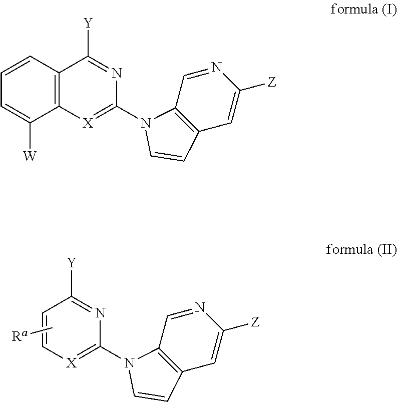

### REFERENCE TO RELATED APPLICATIONS

The present application is based on, and claims priority from, Taiwan application number 110140012 filed Oct. 28, 2021, the disclosure of which is hereby incorporated by reference herein in its entirety.

### TECHNICAL FIELD

The present disclosure relates to a compound for preparation of an imaging agent, preparation methods and medicinal uses thereof, particularly relates to a compound for preparation of microtubule associated Tau protein imaging agent, preparation methods and medicinal uses thereof.

BACKGROUND OF RELATED ARTS

Dementia is a disease phenomenon, not normal aging. The symptoms are not only memory loss but also effects on other cognitive functions, including language, sense of space, calculation, judgment, abstract thinking, attention, etc. The functional degradation of aspects is severe enough to interfere with daily life. The current drugs for dementia have no way to stop or restore damaged brain cells, but they may improve the patient's symptoms or delay the progression of the disease.

Dementia is mainly divided into degenerative dementia, vascular dementia, and cognitive dysfunction caused by other reasons (mixed). Among them, Alzheimer disease's (AD) is the most common, accounting for about 60% of cases of dementia, commonly known as Senile Dementia, which is an irreversible and persistent neurodegenerative disease. Symptoms of Alzheimer's disease include labyrinth, memory loss, cognitive dysfunction, emotional instability, and behavioral changes.

The two most important pathological features of Alzheimer's disease (AD) are abnormal plaques and neurofibrillary tangles in the brain. The plaques are mainly formed by the accumulation of beta amyloid (Aβ), and the neurofibrillary tangles is mainly produced by the hyperphosphorylation of Tau protein, which makes microtubules twisted and deformed and accumulated in brain cells.

At present, the causes of dementia are not clearly understood, so clinical or pre-clinical research has not yet found the most suitable strategy for early diagnosis of Alzheimer's disease.

Tau protein is a kind of microtubule-related protein. Its main function is to bind to microtubules and stabilize brain nerve cells. In recent years, studies have found that changes in the amount of abnormal Tau protein are positively correlated with Alzheimer's disease. In April 2019, the Johns Hopkins University research team even discovered that Tau protein is used as a biomarker. Alzheimer's disease was tested 34 years before the onset. In addition, abnormal Tau protein can also cause rare brain diseases, such as Frontotemporal Dementia (FTD), progressive supranuclear palsy (PSP), cortical basal nucleus degeneration Disease (corticobasal degeneration, CBD), chronic traumatic encephalopathy (CTE) and Pick's disease

(Pick's disease). Therefore, starting from the Tau protein, there is a chance to find a key diagnosis and treatment.

The current dementia diagnosis process will first clarify the medical history and necessary physical, neurological, and mental status examinations to eliminate the possibility of delirium, depression and drugs causing dementia. If it is still suspected to be dementia or the prodromal stage of dementia after the exclusion, a standard inspection process including cognitive function testing (MMSE, CASI, CERAD, etc.), laboratory tests (including complete blood count, biochemical, vitamin B12, vitamin B12, folic acid, syphilis serum, thyroxine, thyroid secretion promoting hormone), head brain tomography and magnetic resonance imaging, etc.

However, according to the above-mentioned procedures, most patients are currently in the middle or late stage at the time of diagnosis. In particular, the diagnosis of Alzheimer's disease has only 80% sensitivity and 70% specificity, and the accuracy of clinical diagnosis is only up to a moderate level.

Therefore, Positron Emission Tomography (PET), which can be used for early diagnosis and high accuracy, is considered to be a powerful tool for the current diagnosis of dementia. After 2012, several positron radiocontrast tracer based on the amyloid hypothesis have been approved by the Food and Drug Aministration (FDA) for marketing, including ¹⁸F-Florbetapir, ¹⁸F-Flutemetamol and

¹⁸F-Florbetaben; but drugs based on the Tau hypothesis are currently only the positron radiocontrast tracer ¹⁸F-AV1451 approved for listing in 2020.

In 2018, the National Dementia Association (NIA-AA) officially included Tau protein into its benchmarks, including the content of Tau protein in cerebrospinal fluid (CSF) and Tau protein positron radiography. However, the acquisition of cerebrospinal fluid samples is an invasive method, which has the risk of infection and patients are prone to discomfort. Therefore, the application potential of Tau positron radiography is worth looking forward to, and many international pharmaceutical companies have already invested in development.

Traditional drug development requires testing and screening tens of thousands of small molecules, and then further synthesizing and testing hundreds of molecules, in order to obtain a few drug candidates suitable for preclinical research; of these, only about one-tenth of drug candidates can pass the final clinical trial. The whole process is slow and costly. It takes an average of ten years and costs billions of dollars. According to a study by Tufts University and data from the US Food and Drug Administration, the average cost of developing new drugs is nearly 2.6 billion U.S. dollars, and it takes 14 years to make the development of new drugs extremely difficult.

At the end of 2016, the Artificial Intelligence report released by Goldman Sachs in the United States: “AI, Machine Learning and Data Fuel the Future of Productivity” mentioned: “With the integration of machine learning and AI, the opportunity exists to significantly derisk the drug discovery and development process, removing $26bn per year in costs, while also driving efficiencies in healthcare information worth more than $28bn per year globally.”

Today, when the big data database is gradually improving, huge information such as small molecule chemical structure and peptide structure will be imported, and machine learning and artificial intelligence technology will be introduced to analyze the above information to verify the relationship between the potential target of the drug and the structure. In the development of chemical small molecule drugs and peptide drugs, it is expected that the drug screening time for pre-drug development can be shortened from 5 to 6 years to 1 to 2 years.

In 2017, the research team of Laboratory of Molecular Biology (LMB) of the Molecular Biology Laboratory of the Medical Research Council (MRC) in England used cryo-EM technology to deconstruct the mystery of the Tau protein structure (reference citations: Fitzpatrick AWP, Falcon B, He S, Murzin AG, Murshudov G , Garringer HJ, Crowther RA, Ghetti B, Goedert M, Scheres SHW. Cryo-EM structures of tau filaments from Alzheimer's disease. Nature. 2017 Jul 13; 547(7662):185-190. doi: 10.1038/nature23002. Epub 2017 Jul 5. PMID: 28678775; PMCID: PMC5552202.).

In the ACS Journal of Chemical Neuroscience published in 2018, the team of KTH Royal Institute of Technology used molecular models to study and discovered various Tau protein positron radiocontrast tracers, including ¹⁸F-FDDNP, ¹⁸F-THK5351, ¹⁸F-THK5117, ¹⁸F-THK5105, ¹⁸F-THK523, ¹⁸F-T808, ¹⁸F-PBB3, etc., can bind to the four sites of Tau fibrils, including Site-1, Site-2, Site-3, and Site-4. Among them, Site-1, Site-3, and Site-4 are called core sites and are located inside the Tau fibrils, and Site-2 is located on the surface of the Tau fibrils, so called surface site. These sites are currently recognized as Tau protein binding sites. The reference Tau fibrils are decamers, consisting of A chain, B chain, C chain, D chain, E chain, F chain, G Chain, H chain, I chain and J chain

(Protein Data Bank in Europe: 5o31, Paired helical filament in Alzheimer's disease brain) (reference citations: Murugan NA, Nordberg A, Agren H. Different Positron Emission Tomography Tau Tracers Bind to Multiple Binding Sites on the Tau Fibril: Insight from Computational Modeling. ACS Chem Neurosci. 2018 Jul. 18; 9(7):1757-1767. doi: 10.1021/acschemneuro.8b00093. Epub 2018 May 2. PMID:

The Synapse journal published in 2020 also confirmed that ¹²⁵I-IPPI not only binds to the four recognized binding sites of Tau fibrils during computer simulation, but also can be applied to the positron radiography of Tau protein. Therefore, the application of computer simulation technology quickly verifies whether the compound and tubulin have potential for binding, which can accelerate the speed of drug screening and reduce the cost of drug screening. (Mukherjee J, Liang C, Patel KK, Lain PQ, Mondal R. Development and evaluation of [¹²⁵I]IPPI for Tau imaging in postmortem human Alzheimer's disease brain. Synapse. 2021 January; 75(1):e22183. doi: 10.1002/syn.22183. Epub 2020 Aug 6. PMID: 32722889; PMCID: PMC8373522.)

Tau protein positron radiocontrast tracer is a non-invasive way to identify the number and distribution of Tau fibrillary tangles in the brain to identify possible patients. At the same time, it also allows drug researchers to evaluate the biochemical changes or metabolic effects of candidate drugs in the body for a long time to find potential Tau protein therapeutic drugs.

The development of positron radiocontrast tracers using Tau protein as a biological indicator is in full swing. These drugs include positron radiocontrast tracers: ¹⁸FDDNP, ¹¹C-PBB3, ¹⁸F-THK5351, ¹⁸F-THK5117, ¹⁸F-THK5105, ¹⁸F-THK523, ¹⁸F-THK5117, ¹⁸F-AV1451, Lansoprazole series and ¹⁸F-T808, etc., and the recent development of ¹⁸F-MK6240, ¹⁸F-GTP1, ¹⁸F-APN-1607,

¹⁸F-PI-2620, ^{I}T-JNJ-067, and ¹⁸F-RO-948 (¹⁸F-RO6958948) and so on.

At present, the only drug approved by the FDA is the positron radiocontrast tracer ¹⁸F-AV1451, and the drugs that are in the second phase of clinical trials are ¹⁸F-MK6240, ¹⁸F-APN-1607, ¹⁸F-THK5351, ¹⁸F-PI-2620 and ¹⁸F-GTP1, the drugs that have been in the first phase of clinical trials include ¹⁸F-FDDNP, ¹⁸F-R06958948, ¹⁸F-JNJ-067, and ¹⁸F-T808. Since there is currently only one approved drug for the Tau protein tracer, and many international pharmaceutical companies have already invested in development, it can be seen that its application potential is worth looking forward to and continuous tracking. Although Tau protein positron radiography faces many challenges, such as ¹⁸F-DDNP is the first positron radiocontrast tracer developed for both Tau protein and beta amyloid plaque binding imaging, but it lacks in vivo specificity and selectivity.

At present, Tau protein positron radiocontrast tracers may deviate from the target absorption (off-target), for example, the absorption of ¹¹C-PBB3 and ¹⁸F-AM-PBB3 in the venous sinus are significantly higher relative to ¹⁸F-PI-2620.

Other Tau protein positron radiocontrast tracers such as ¹⁸F-THK-5351, ¹⁸F-THK-5317 or ¹T-AV1451 have higher striatal uptake than ¹⁸F-PI-2620 and ¹⁸F-MK-6240 in normal brain imaging. In addition, the tracer ¹⁸F-RO-948 has also been reported to have less deviation from the target absorption in known absorption sites (such as the choroid plexus and subcortical gray matter structure) compared with ¹⁸F-AV1451.

### SUMMARY

One purpose of the present disclosure is to solve the problems of insufficient specificity and insufficient selectivity of the current Tau protein positron radiocontrast tracer.

According to the purpose of the present disclosure, the present disclosure provides a compound shown as formula (I) and formula (II), which is used to prepare a radiographic tracer that can target binding to Tau protein. The structure of the tracer is designed with different structures based on the existing Tau protein positron radiocontrast tracer (¹⁸F-MK6240) to increase specificity and selectivity in vivo.

In one aspect, the present disclosure provides a compound shown as formula (I), X is CH or N; Y is I or H; Z is NH₂ or H; W is halogen or H;

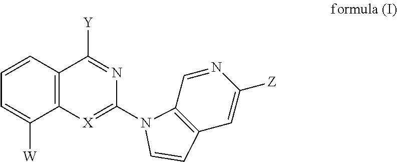

In one aspect, the present disclosure provides a compound shown as formula

(II), X is CH or N; Y is I or H; Z is NH₂ or H; Ra is C₁₋₆ alkyl, C₂₋₆ alkenyl, C₂₋₆ alkynyl, C₁₋₆ haloalkyl, C₂₋₆ haloalkynyl, (CH₂)₁₋₆ pyrazolyl, C₁₋₆ alkylboranyl, and C₂₋₆ alkenylboranyl, or combinations of any one of the above-mentioned.

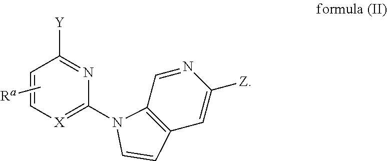

In one aspect, the compound is shown as formula (I), wherein the X position is CH, the Y position is I or H, the Z position is H, and the W position is F, and the structure of the compound is as follows:

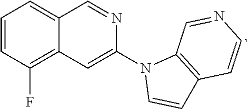 [SMILES: Fc1cccc2cnc(-n3ccc4ccncc43)cc12]

and the compound is named as AI-INER-M-1.

In one aspect, the compound is shown as formula (I), wherein the X position is CH, the Y position is H, the Z position is NH₂, and the W position is H, and the structure of the compound is as follows:

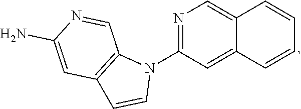 [SMILES: Nc1cc2ccn(-c3cc4ccccc4cn3)c2cn1]

and the compound is named as AI-INER-M-2.

In one aspect, the compound is shown as formula (II), wherein the X position is CH, the Y position is H, the Z position is H, and the Ra position is fluoromethyl and ethynyl, and the structure of the compound is as follows:

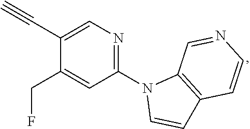 [SMILES: C#Cc1cnc(-n2ccc3ccncc32)cc1CF]

and compound is named as AI-INER-M-3.

In one aspect, the compound is shown as formula (II), wherein the X position is N, the Y position is H, the Z position is H, and the Ra position is propylpyrazolyl, and the structure of the compound is as follows:

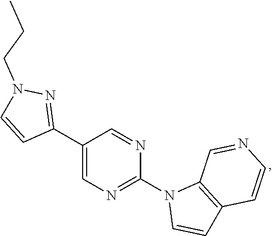 [SMILES: CCCn1ccc(-c2cnc(-n3ccc4ccncc43)nc2)n1]

and the compound is named as AI-INER-M-4.

In one aspect, the compound is shown as formula (II), wherein the X position is CH, the Y position is H, the Z position is H, and the Ra position is Piperidin-4-yl, and the structure of the compound is as follows:

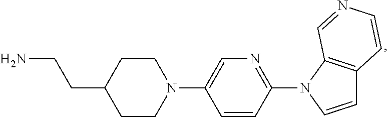 [SMILES: NCCC1CCN(c2ccc(-n3ccc4ccncc43)nc2)CC1]

and the compound is named as AI-INER-M-5.

In one aspect, the compound is shown as formula (II), wherein the X position is CH, the Y position is I, the Z position is H, and the Ra position is pyridyl, and the structure of the compound is as follows:

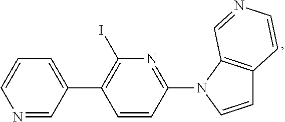 [SMILES: Ic1nc(-n2ccc3ccncc32)ccc1-c1cccnc1]

and the compound is named as AI-INER-M-6.

In one aspect, the present disclosure provides a method for preparing compound AI-INER-M-1, which includes the following steps:

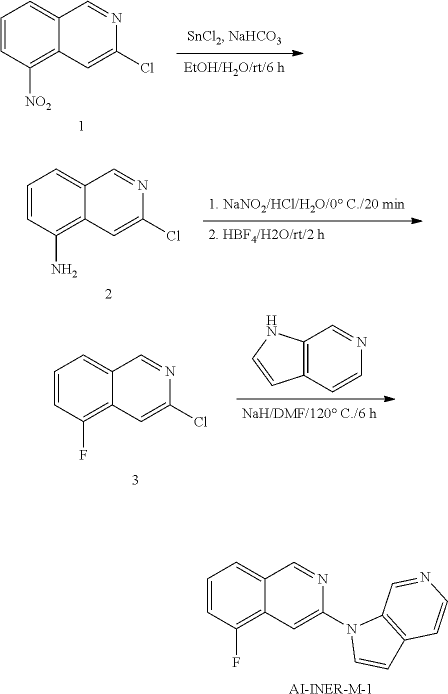

dissolving Compound 1 (3-chloro-5-nitroisoquinoline) in ethanol (EtOH) and double-distilled water (ddH₂O), adding stannous chloride (SnC1₂) and sodium bicarbonate (NaHCO₃), and performing reaction at room temperature for 6 hours (the number of mole of Compound 1:the number of mole of SnCl₂:the number of mole of NaHCO₃=1:2:2), and then performing concentration under reduced pressure and purification to obtain Compound 2 (3-chloroisoquinolin-5-amine); then, dissolving Compound 2 (3-chloroisoquinolin-5-amine) and sodium nitrite (NaNO₂) in 1N hydrochloric acid (HCl), performing reaction under nitrogen for 0° C. for 20 minutes, and adding fluoroboric acid (HBF₄) then returning to room temperature to perform reaction for 2 hours (the number of mole of Compound Compound 2:the number of mole of NaNO₂:the number of mole of HBF₄=1:2:2), and then performing concentration under reduced pressure and purification to obtain Compound 3 (3-chloro-5-fluoroisoquinoline); adding Compound 3 with indole and NaH to be dissolved in dimethylformamide (DMF) and performing reaction at 120° C. for 6 hours (the number of mole of the compound 3:the number of mole of indole:the number of mole of NaH=1:1.5:1.5), and at last performing extraction and purification to obtain compound AI-INER-M-1 (5-fluoro-3-(1H-pyrrolo [2,3-c]pyridin-1-yl)isoquinoline).

In one aspect, the present disclosure provides a method for preparing compound AI-INER-M-2, which includes the following steps:

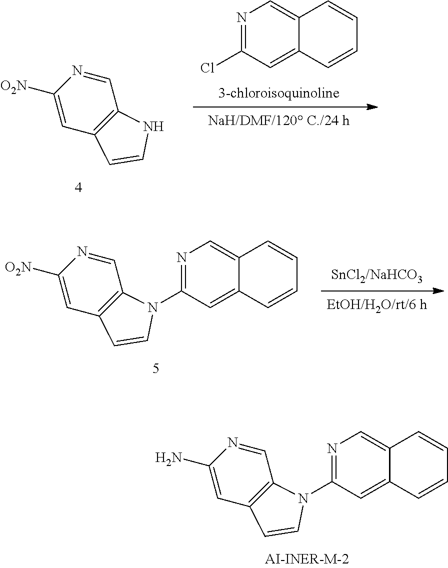 [SMILES: Clc1cc2ccccc2cn1 ; O=[N+]([O-])c1cc2cc[nH]c2cn1 ; O=[N+]([O-])c1cc2ccn(-c3cc4ccccc4cn3)c2cn1 ; Nc1cc2ccn(-c3cc4ccccc4cn3)c2cn1]

dissolving Compound 4 (5-nitro-1H-pyrrolo[2,3-c]pyridine) in DMF and adding 3-chloroisoquinoline and NaH to perform reaction at 120° C. for 24 hours (the number of mole of Compound 4:the number of mole of 3-chloroisoquinoline:the number of mole of NaH=1:1.5:1.5), and then performing extraction and purification to obtain Compound 5 (3-(5-nitro-1H-pyrrolo [2,3-c]pyridin-1-yl)isoquinoline); dissolving Compound 5 in EtOH and ddH₂O, and adding SnCl₂ and NaHCO₃ to perform reaction for 6 hours at room temperature (the number of mole of Compound 5:the number of mole of SnCl₂:the number of mole of NaHCO₃=1:1.5:1.5), and at last performing extraction and purification to obtain compound AI-INER-M-2 (1-(isoquinolin-3-yl)-1H-pyrrolo [2,3-c]pyridin-5-amine).

In one aspect, the present disclosure provides a method for preparing compound AI-INER-M-3, which includes the following steps:

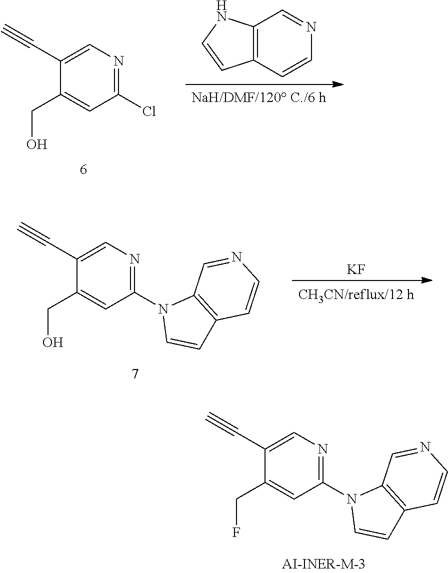 [SMILES: C#Cc1cnc(Cl)cc1CO ; c1cc2cc[nH]c2cn1 ; C#Cc1cnc(-n2ccc3ccncc32)cc1CO ; "KF" ; C#Cc1cnc(-n2ccc3ccncc32)cc1CF]

dissolving Compound 6 (2-chloro-5-ethynylpyridin-4-yl)methanol) in DMF and adding indole and NaH to perform reaction at 120° C. for 6 hours (the number of mole of Compound 6:the number of mole of indole:the number of mole of NaH =1:1.5:1.5), and then performing extraction and purification to obtain Compound 7 ([5-ethynyl-2-(1H-pyrrolo [2,3-c]pyridin-1-yl)pyridin-4-yl]methanol); dissolving Compound 7 and potassium fluoride (KF) in acetonitrile (CH₃CN), and heating to a high temperature at reflux with introducing nitrogen gas to perform reaction for 12 hours (the number of mole of Compound 7:the number of mole of KF=1:2), and at last performing concentration under reduced pressure and purification to obtain compound AI-INER-M-3 (1-[5-ethynyl-4-(fluoromethyl)pyridin-2-yl]-1H-pyrrolo[2,3-c] pyridine).

In one aspect, the present disclosure provides a method for preparing compound AI-INER-M-4, which includes the following steps:

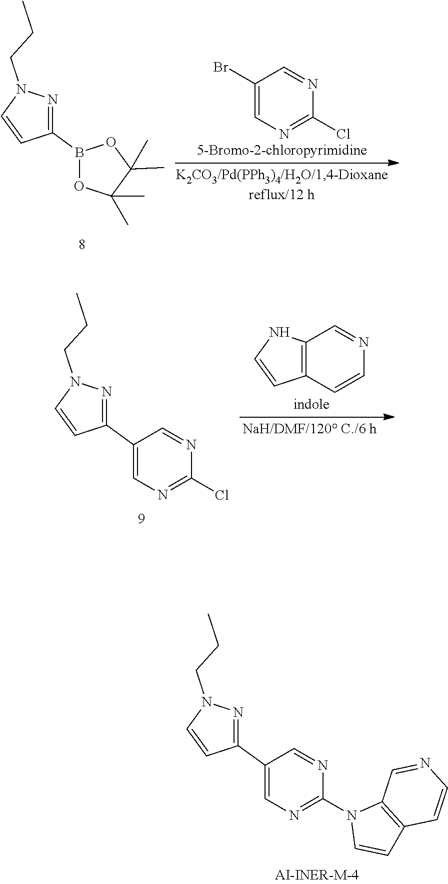 [SMILES: CCCn1ccc(B2OC(C)(C)C(C)(C)O2)n1 ; Clc1ncc(Br)cn1 ; CCCn1ccc(-c2cnc(Cl)nc2)n1 ; c1cc2cc[nH]c2cn1 ; CCCn1ccc(-c2cnc(-n3ccc4ccncc43)nc2)n1]

dissolving Compound 8 (1-Propyl-3-(4,4,5,5-tetramethyl-1,3,2-dioxaborolan-2-yl)pyrazole) and 5-bromo-2-chloropyrimidine in a mixture solution of H₂O and 1,4-dioxane with introducing nitrogen gas, and adding potassium carbonate (K₂CO₃) and tetrakis(triphenylphosphine)palladium (Pd(PPh₃)₄), heating at reflux to perform reaction for 12 hours (the number of mole of Compound 8:the number of mole of 5-bromo-2-chloropyrimidine: the number of mole of K₂CO₃ :the number of mole of Pd(PPh₃)₄)=1:1:2:0.05, and then performing concentration under reduced pressure and purification to obtain Compound 9 (2-chloro-5-(1-propyl-1H-pyrazol-3-yl)pyrimidine); adding Compound 9 with indole and NaH to be dissolved in DMF and reacted at 120° C. for 6 hours (the number of mole of Compound 9:the number of mole of indole:the number of mole of NaH=1:1.5:1.5), and then perforing concentration under reduced pressure and purification, and at last obtaining compound AI-INER-M-4 (1-[5-(1-propyl-1H-pyrazol-3-yl)pyrimidin-2-yl]-1H-pyrrolo [2 ,3-c]pyridine).

In one aspect, the present disclosure provides a method for preparing compound AI-INER-M-5, which includes the following steps:

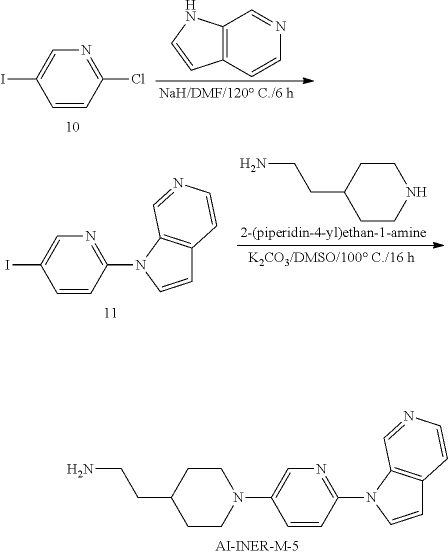 [SMILES: Clc1ccc(I)cn1 ; c1cc2cc[nH]c2cn1 ; Ic1ccc(-n2ccc3ccncc32)nc1 ; NCCC1CCNCC1 ; NCCC1CCN(c2ccc(-n3ccc4ccncc43)nc2)CC1]

dissolving Compound 10 (2-chloro-5-iodopyridine) in DMF, adding indole and NaH to perform reaction at 120° C. for 6 hours (the number of mole of Compound 10:the number of mole of indole:the number of mole of NaH=1:1.5:1.5), and then performing extraction and purification to obtain Compound 11 (1-(5-iodopyridin-2-yl)-1H-pyrrolo [2,3-c]pyridine); dissolving Compound 11 in dimethyl sulfoxide (DMSO) and adding 2-(piperidin-4-yl)ethan-1-amine and K₂CO₃, heating to a temperature of 100° C. to perform reaction for 16 hours (the number of mole of Compound 11:the number of mole of 2-(piperidin-4-yl)ethan-1-amine:the number of mole of K₂CO₃=1:1.5:1.5), and at last perforing extraction and purification to obtain compound AI-INER-M-5 (2-{1-[6-(1H-pyrrolo [2,3-c]pyridin-1-yl)pyridin-3-yl]piperidin-4-yl}ethan-1-amine).

In one aspect, the present disclosure provides a method for preparing compound AI-INER-M-6, which includes the following steps:

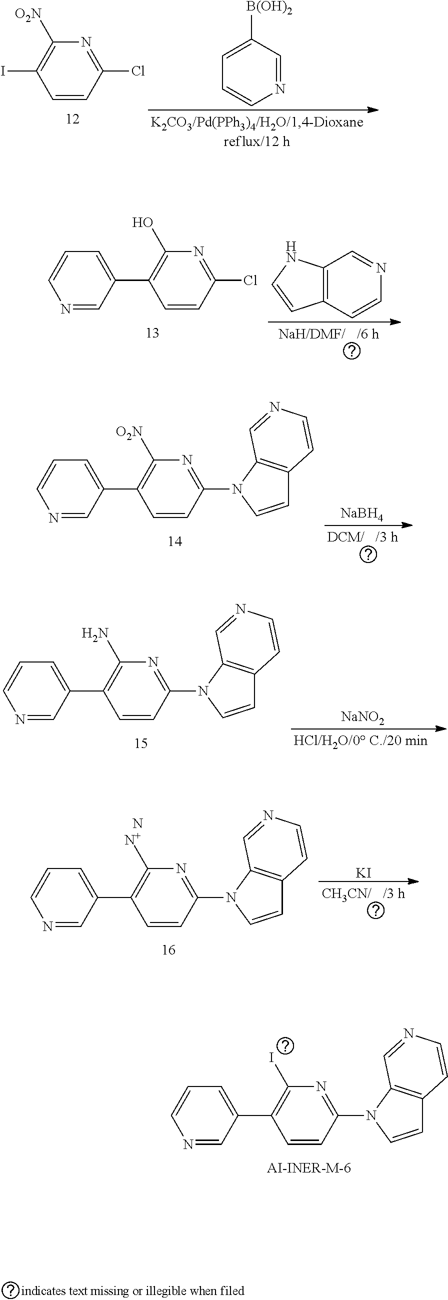

dissolving Compound 12 (6-chloro-3-iodo-2-nitropyridine) in a mixture solution of ddH₂O and 1,4-dioxane, and adding 3-pyridinylboronic acid, K₂CO₃ and Pd(PPh₃)₄, heating at reflux to perform reaction for 12 hours (the number of mole of Compound 12:the number of mole of 3-pyridinylboronic acid: the number of mole of K₂CO:the number of mole of Pd(PPh₃)₄=1:1:2:0.05, and then performing extraction and purification to obtain Compound 13 (6-chloro[3,3′-bipyridin]-2-ol); dissolving Compound 13 in DMF and adding indole and NaH to perform reaction at room temperature for 6 hours (the number of mole of Compound 13:the number of mole of indole:the number of mole of NaH=1:1.5:1.5), and then performing extraction and purification, and at last obtaining Compound 14 (2-nitro-6-(1H-pyrrolo [2,3-c]pyridin-1-yl)-3,3′-bipyridine); dissolving Compound 14 in dichloromethane (DCM) and adding sodium borohydride (NaBH₄) to react at room temperature for 3 hours (the number of mole of Compound 14:the number of mole of NaBH₄=1:1.5), and at last performing extraction and purification to obtain Compound 15 (6-(1H-pyrrolo[2,3-c]pyridin-1-yl)[3,3′-bipyridin]-2-amine); dissolving Compound 15 in 1N HC1 and adding NaNO₂, performing reaction of the mixture under nitrogen gas at 0° C. for 20 minutes (the number of mole of Compound 15:the number of mole of NaNO₂=1:1.5), and obtaining Compound 16 (6-(1H-pyrrolo [2,3-c]pyridin-1-yl) [3,3′-bipyridine]-2-diazonium), draining Compound 16 and directly proceeding the next reaction, adding potassium iodide (KI) and CH₃CN then performing reaction at room temperature for 3 hours (the number of mole of Compound 16:the number of mole of KI=1:2), and the iodine in KI is selected from the group consisting of 123I, ¹²⁴I, 127I and ¹³¹I and at last performing concentration under reduced pressure and purification to obtain compound AI-INER-M-6 (2-iodo-6-(1H-pyrrolo [2,3-c]pyridin-1-yl)-3,3′-bipyridine).

In one aspect, the present disclosure provides a use of the compound shown as formula (I) for the imaging of Tau protein.

In one aspect, the present disclosure provides a use of the compound shown as formula (II) for the imaging of Tau protein.

The compound shown as formula (I) or formula (II) shown in the present disclosure can improve the problems of insufficient specificity and insufficient selectivity of existing Tau protein positron radiocontrast tracers.

Furthermore, in the application provided by the present disclosure, it may also have such characteristics:the tumor , including, but not limited to, liver cancer, lung cancer, primary colorectal cancer, cervical squamous cell carcinoma, gastric cancer, prostate cancer, and lung adenocarcinoma.

### BRIEF DESCRIPTION OF THE DRAWINGS

FIG. 1 is a graph showing the force analysis of the compound AI-INER-M-5 at Site-2 of Tau fibril.

### DETAILED DESCRIPTIONS OF EMBODIMENTS

The present disclosure is an artificial intelligence model based on a variational auto-encoder (Variational Auto-Encoder, VAE) imported with the small molecule chemical structure information obtained from the small molecule compound database (ZINC database, ChEMBL database and Drugbank database), to produce a series of compounds that can bind to Tau protein, including AI-INER-M-1, AI-INER-M-2, AI-INER-M-3, AI-INER-M-4, AI-INER-M-5 and AI-INER-M-6.

The physical and chemical properties of the compound proposed in the present disclosure are in line with the current common physical and chemical properties of central nerve drugs including the logarithmic value of the lipid-water partition coefficient (log P) is between 2 and 5, the molecular weight is ≤450, and the topological polar surface area (TPSA) is <90 ⁵²¹I, the number of hydrogen bond donors is less than 3, the number of hydrogen bond acceptors is less than 7, and the number of rotatable bonds is between 0-8.

In some embodiments, the present disclosure provides a compound shown as formula (I):

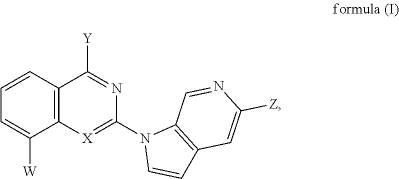

wherein X is CH or N; Y is I or H; Z is NH₂ or H; W is halogen or H.

In some embodiments, the present disclosure provides a compound shown as formula (II):

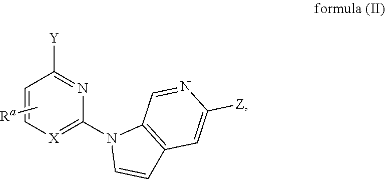

wherein, X is CH or N; Y is I or H; Z is NH₂ or H; Ra is C₁₋₆ alkyl, C₂₋₆ alkenyl, C₂₋₆ alkynyl, C₁₋₆ haloalkyl, C₂₋₆ haloalkynyl, (CH₂)₁₋₆ pyrazolyl, C₁₋₆ alkylboranyl, and C₂₋₆ alkenylboranyl, or combinations of any one of the above-mentioned.

In some embodiments of the present disclosure, there is provided a use of the compound shown as formula (I) for the imaging of Tau protein.

In some embodiments of the present disclosure, there is provided a use of the compound shown as formula (II) for the imaging of Tau protein.

Hereinafter, the preparation method of the compound shown as formula (I) or formula (II) of the present disclosure will be illustrated by examples and drawings. However, the examples and drawings provided below are exemplary and are intended to explain the content of the present disclosure. It is not a limitation of the present disclosure.

### EXAMPLE 1

### Preparation Method of Compound AI-INER-M-1

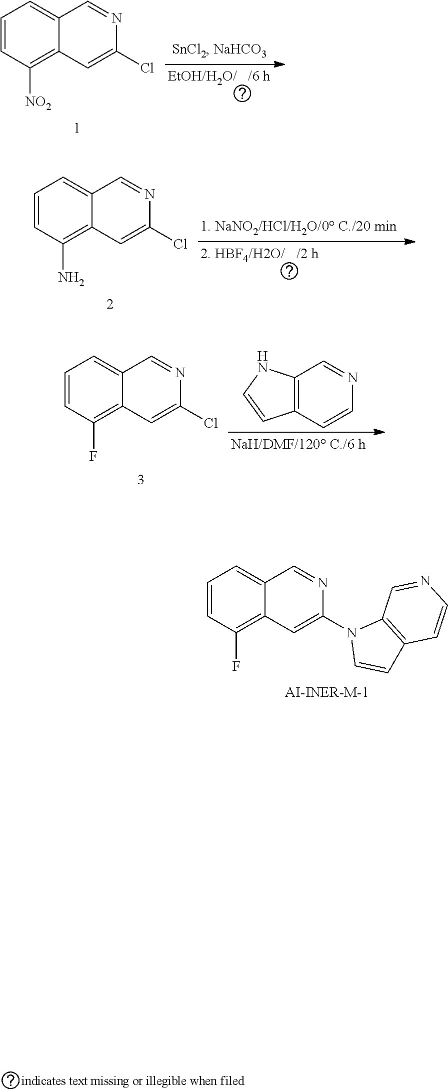

Compound 1 (3-chloro-5-nitroisoquinoline) was dissolved in EtOH and ddH₂O and added with SnCl₂ and NaHCO₃ (the number of mole of the compound 1:the number of mole of SnCl₂:the number of mole of NaHCO₃=1:2:2), the reaction was performed at room temperature for 6 hours to form an intermediate, and then the intermediate was concentrated under reduced pressure and purified to obtain compound 2 (3-chloroisoquinolin-5-amine). Compound 2: MS (m/z) =179.03 [M+H]+.

Compound 2 and NaNO₂ were dissolved in IN HC1, and then reacted under nitrogen gas at 0° C. for 20 minutes, and HBF₄ was added, returned to room temperature and reacted for 2 hours (the number of mole of Compound 2:the number of mole of NaNO₂:the number of mole of HBF₄=1:2:2) to form an intermediate, and then the intermediate was concentrated under reduced pressure and purified to obtain Compound 3 (3-chloro-5-fluoroisoquinoline); Compound 3: MS (m/z)=182.01[M+H]+.

Compound 3 was added with indole and NaH and dissolved in DMF, and reacted at 120° C. for 6 hours (the number of mole of Compound 3:the number of mole of indole:the number of mole of NaH=1:1.5:1.5) to form an intermediate, and then the intermediate was extracted and purified to obtain compound AI-INER-M-1 (5-fluoro-3-(1H-pyrrolo [2,3-c]pyridin-1-yl)isoquinoline); compound AI-INER-M-1: MS (m/z)=264.09 [M+H]³⁰ .

### EXAMPLE 2

### Preparation Method of Compound AI-INER-M-2

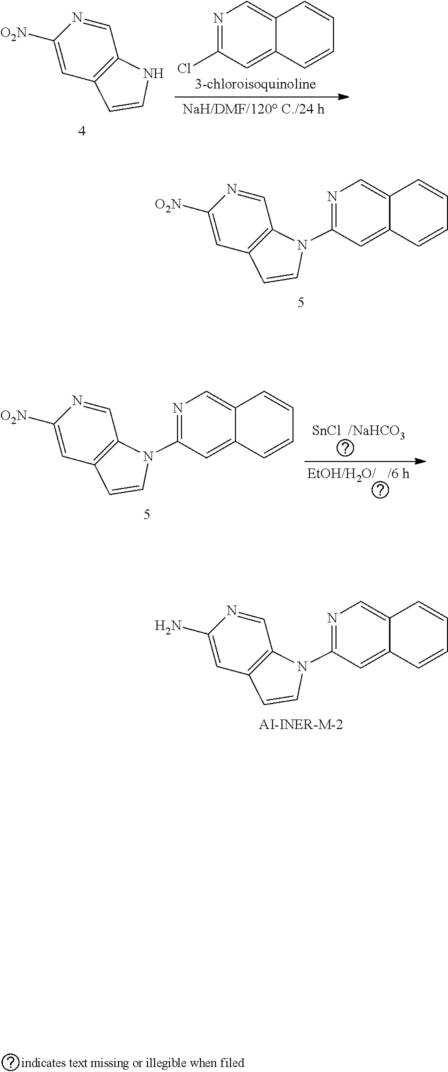

Compound 4 (5-nitro-1H-pyrrolo[2,3-c]pyridine) was dissolved in DMF and 3-chloroisoquinoline and NaH were added, and reacted at 120° C. for 24 hours (the number of mole of Compound 4:the number of mole of 3-chloroisoquinoline:the number of mole of NaH=1:1.5:1.5) to form an intermediate, and then the intermediate was extracted and purified to obtain Compound 5 (3-(5-nitro-1H-pyrrolo[2,3-c]pyridin-1-yl)isoquinoline); Compound 5: MS (m/z)=291.08 [M+H]⁺.

Compound 5 was dissolved in EtOH and ddH₂O and added with SnCl₂ and NaHCO₃, and reacted at room temperature for 6 hours (the number of mole of Compound 5:the number of mole of SnCl₂:the number of mole of NaHCO₃=1:1.5:1.5) to form an intermediate, and then the intermediate was extracted and purified to obtain compound AI-INER-M-2 (1-(isoquinolin-3-yl)-1H-pyrrolo [2,3-c] pyridin-5-amine); compound AI-INER-M-2: MS (m/z)=261.11 [M+H]⁺.

### EXAMPLE 3

### Preparation Method of Compound AI-INER-M-3

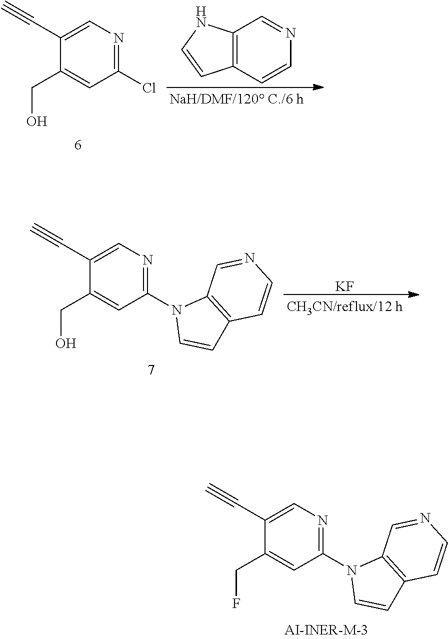 [SMILES: C#Cc1cnc(Cl)cc1CO ; c1cc2cc[nH]c2cn1 ; C#Cc1cnc(-n2ccc3ccncc32)cc1CO ; "KF" ; C#Cc1cnc(-n2ccc3ccncc32)cc1CF]

Compound 6 (2-chloro-5-ethynylpyridin-4-yl)methanol) was dissolved in DMF and added with indole and NaH, and reacted for 6 hours at 120° C. (the number of mole of Compound 6:the number of mole of indole:the number of mole of NaH=1:1.5:1.5) to form an intermediate, and then the intermediate was extracted and purified to obtain Compound 7 ([5-ethynyl-2-(1H-pyrrolo [2,3-c] pyridin-1-yl)pyridin-4-yl]methanol); Compound 7: MS (m/z)=250.09 [M+H]+.

Compound 7 and KF were dissolved in CH₃CN, and reacted for 12 hours by heating to a high temperature at reflux with introducing nitrogen gas (the number of mole of Compound 7:the number of mole of of KF=1:2) to form an intermediate, and then the intermediate was concentrated under reduced pressure and purified to obtain compound AI-INER-M-3(1[5-ethynyl-4-(fluoromethyl) pyridin-2-yl]-1H-pyrrolo [2,3-c] pyridine); compound M-INER-M-3: MS (m/z) =252.09 [M+H]⁺.

### EXAMPLE 4

### Preparation Method of Compound AI-INER-M-4

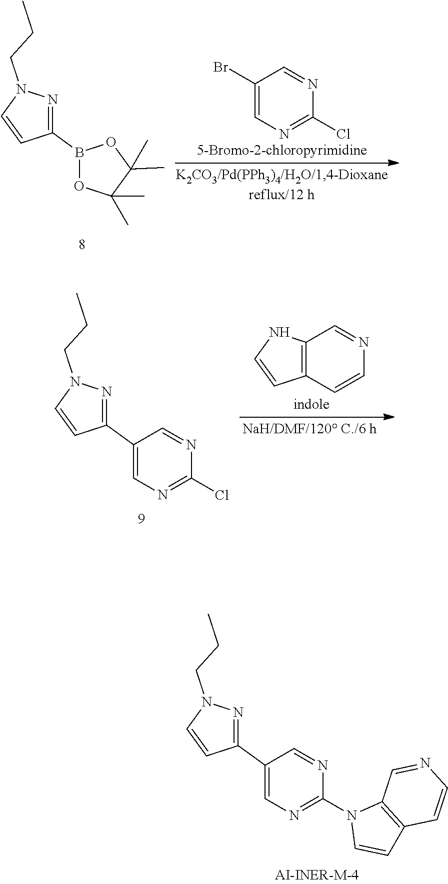 [SMILES: CCCn1ccc(B2OC(C)(C)C(C)(C)O2)n1 ; Clc1ncc(Br)cn1 ; CCCn1ccc(-c2cnc(Cl)nc2)n1 ; c1cc2cc[nH]c2cn1 ; CCCn1ccc(-c2cnc(-n3ccc4ccncc43)nc2)n1]

Compound 8 (1-Propyl-3-(4,4,5,5-tetramethyl-1,3,2-dioxaborolan-2-yl)pyra-zole) and 5-Bromo-2-chloropyrimidine dissolved in a mixure solution of ddH₂O and 1,4-dioxane with introducing nitrogen gas, K₂CO₃ and Pd(PPh₃)₄ were added, heated at reflux, and then reacted for 12 hours (the number of mole of Compound 8:the number of mole of 5-bromo-2-chloropyrimidine: the number of mole of K₂CO₃:the number of mole of of Pd(PPh₃)₄=1:1:2:0.05) to form an intermediate, and then the intermediate was concentrated under reduced pressure and purified to obtain Compound 9 (2-chloro-5-(1-propyl-1H-pyrazol-3-yl)pyrimidine); Compound 9: MS (m/z) 223.07 [M+H]+.

Compound 9 was added with indole and NaH and dissolved in DMF, and reacted at 120° C. for 6 hours (the number of mole of Compound 9:the number of mole of indole:the number of mole of NaH=1:1.5:1.5 to form an intermediate, and then the intermediate was concentrated under reduced pressure and purified to obtain compound AI-INER-M-4 (1-[5-(1-propyl-1H-pyrazol-3-yl)pyrimidin-2-yl]-1H-pyrrolo [2,3-c]pyridine; compound AI-INER-M-4: MS (m/z) 305.15 [M+H]⁺.

### EXAMPLE 5

### Preparation Method of Compound AI-INER-M-5

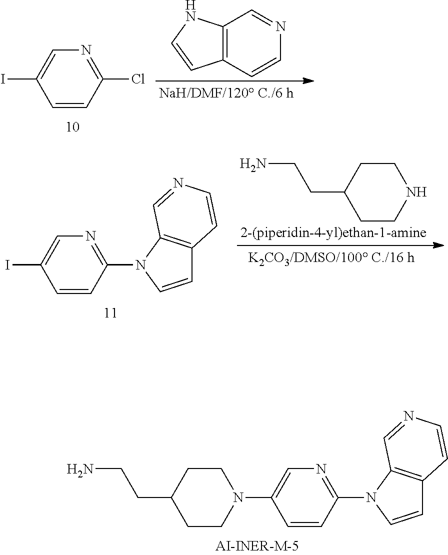 [SMILES: Clc1ccc(I)cn1 ; c1cc2cc[nH]c2cn1 ; Ic1ccc(-n2ccc3ccncc32)nc1 ; NCCC1CCNCC1 ; NCCC1CCN(c2ccc(-n3ccc4ccncc43)nc2)CC1]

Compound 10 (2-chloro-5-iodopyridine) was dissolved in DMF and added with indole and NaH, and reacted at 120° C. for 6 hours (the number of mole of compound 10:the number of mole of indole:the number of mole of NaH=1:1.5:1.5) to form an intermediate, and then the intermediate was extracted and purified to obtain compound 11 (1-(5-iodopyridin-2-yl)-1H-pyrrolo[2,3-c]pyridine); Compound 11: MS (m/z)=321.98 [M+H]+.

Compound 11 was dissolved in DMSO, 2-(piperidin-4-yl)ethan-1-amine and K₂CO₃ were added, heated to 100° C. and then reacted for 16 hours (the number of mole of Compound ll:the number of mole of 2-(piperidin-4-yl)ethan-1-amine:the number of mole of K₂CO₃=1:1.5:1.5) to form an intermediate, and then the intermediate was extracted and purified to obtain compound AI-INER-M-5 (2-{1-[6-(1H-pyrrolo [2,3-c]pyridin-1-yl)pyridin-3-yl]piperidin-4-yl}ethan-1-amine); compound AI-INER-M-5: MS (m/z) 322.20 [M+H]⁺.

### EXAMPLE 6

### Preparation Method of Compound AI-INER-M-6

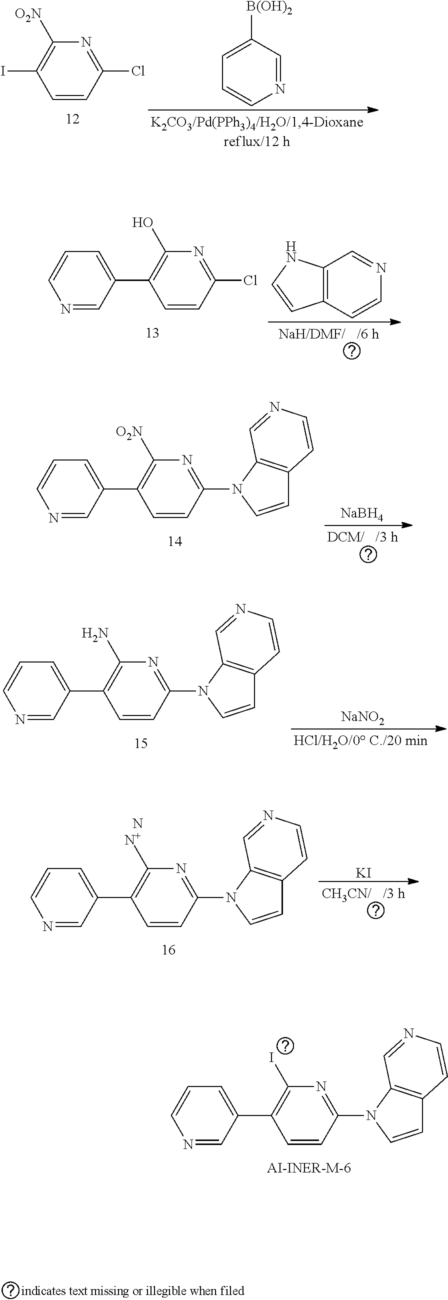

Compound 12 (6-chloro-3-iodo-2-nitropyridine) was dissolved in a mixture solution of ddH₂O and 1,4-dioxane, and 3-pyridinylboronic acid, K₂CO₃ and Pd(PPh₃)₄ were added, and then heated at reflux to react for 12 hours (the number of mole of Compound 12:the number of mole of 3-pyridinylboronic acid:the number of mole of K₂CO₃:the number of mole of Pd(PPh₃)₄=1:1:2:0.05) to form an intermediate, and then the intermediate was extracted and purified to obtain compound 13 (6-chloro[3,3′-bipyridin]-2-ol); Compound 13: MS (m/z)=207.03 [M+H]⁺.

Compound 13 was dissolved in DMF and added with indole and NaH, and reacted at room temperature for 6 hours (the number of mole of Compound 13:the number of mole of indole:the number of mole of NaH=1:1.5:1.5)) to form an intermediate, and then the intermediate was extracted and purified to obtain Compound 14 (2-nitro-6-(1H-pyrrolo [2,3-c]pyridin-1-yl)-3,3′-bipyridine); Compound 14: MS (m/z)=318.09 [M+H]+.

Compound 14 was dissolved in DCM and added with NaBH₄, and reacted at room temperature for 3 hours (the number of mole of compound 14:the number of mole of NaBH₄=1:1.5) to form an intermediate, and then the intermediate was extracted and purified to obtain compound 15 (6-(1H-pyrrolo[2,3-c]pyridin-1-yl) [3,3′-bipyridin]-2-amine); Compound 15: MS (m/z)=288.12 [M+H]+.

Compound 15 was dissolved in IN HC1 and added with NaNO₂. The mixture was reacted under nitrogen at 0° C. for 20 minutes (the number of mole of compound 15:the number of mole of NaNO₂=1:1.5) to obtain compound 16 (6-(1H-pyrrolo [2,3-c]pyridin-1-yl) [3,3 ′-bipyridine] -2-diazonium), then Compound 16 was drained and directly proceeded in the next step, and KI and CH₃CN were added to react at room temperature for 3 hours (the number of mole of Compound 16:the number of mole of KI=1:2) to form an intermediate, wherein the iodine (I) in KI was selected from the group consisting of ¹²³I, ¹²⁴I, ¹²⁷I and ¹³¹I, and then the intermediate was extracted and purified to obtain compound AI-INER-M-6 (2-iodo-6-(1H-pyrrolo [2,3-c]pyridin-1-yl)-3,3′-bipyridine); wherein iodine was ¹²³1, compound AI-INER-M-6: MS (m/z)=395.01 [M+H]⁺; wherein iodine was ¹²⁴I, M-INER-M-6: MS (m/z)=396.01 [M+H]⁺; wherein iodine was ¹²⁷I, compound M-INER-M-6: MS (m/z) =399.01 [M+H]⁺; wherein iodine was ¹³¹I, compound M-INER-M-6: MS (m/z)=403.01 [M+H]⁺.

Small-molecule compound docking experiment:

Small molecule compound docking experiments was conducted using the Flexible Docking module of the biomacromolecule computing simulation platform (BIOVIA Discovery Studio) for drug discovery, to confirm the intensity of the interaction force of each of the 6 compounds, such as M-INER-M-1, AI-INER-M-2 AI-INER-M-3, AI-INER-M-4, AI-INER-M-5, and AI-INER-M-6, when binding at the four binding sites of Tau fibrils. The four binding sites of Tau fibrils include “Site-1”, “Site-2”, “Site-3” and “Site-4”, which are based on the definition of the four binding sites of Tau protein in internationally recognized journals (reference citations: Mukherjee, J., Liang, C., Patel, K. K., Lain, P. Q., Mondal, R. Development and evaluation [¹²⁵I]IPPI for tau imaging in post-mortem human Alzheimer's disease brain. Synapse, 75: e22183, 2021). Based on the docking score of CDOCKER INTERACTION ENERGY, the docking score of CDOCKER INTERACTION ENERGY represented the intensity of the interaction force between protein and ligand. The unit was “kcal/mole”, wherein ¹⁸F-MK6240 was the second generation Tau protein positron radiocontrast tracer, which was used as the control group of this experiment, hereinafter referred to as “MK6240”; the results are shown in Table 1.

TABLE 1

| Compound | Site-1 | Site-2 | Site-3 | Site-4 |
|---|---|---|---|---|
| MK6240 | 27.8 | 38.1 | 35.2 | 34.6 |
| AI-INER-M-1 | 26.2 | 38.1 | 33.4 | 33.8 |
| AI-INER-M-2 | 32.1 | 33.0 | 35.8 | 35.8 |
| AI-INER-M-3 | 25.4 | 38.0 | 33.7 | 38.4 |
| AI-INER-M-4 | 35.4 | 51.5 | 43.6 | 43.0 |
| AI-INER-M-5 | 39.2 | 61.0 | 51.8 | 46.7 |
| AI-INER-M-6 | 33.7 | 39.6 | 40.8 | 39.3 |

In this embodiment, the docking score of CDOCKER INTERACTION ENERGY of the compound MK6240 at the four sites of Tau fibrils were shown that Site-1 was 27.8 kcal/mole, Site-2 was 38.1 kcal/mole, Site-3 was 35.2 kcal/mole, and Site -4 was 34.6 kcal/mole.

In this embodiment, the docking score of CDOCKER INTERACTION ENERGY of the compound AI-INER-M-1 at 4 sites of Tau fibrils were shown that Site-1 was 26.2 kcal/mole, Site-2 was 38.1 kcal/mole, Site-3 was 33.4 kcal/mole, and Site -4 was 33.8 kcal/mole.

In this embodiment, the docking score of CDOCKER INTERACTION ENERGY of the compound AI-INER-M-2 at 4 sites of Tau fibrils were shown that Site-1 was 32.1 kcal/mole, Site-2 was 33.0 kcal/mole, Site-3 was 35.8 kcal/mole, and Site -4 was 35.8 kcal/mole.fr

In this embodiment, the docking score of CDOCKER INTERACTION ENERGY of the compound AI-INER-M-3 at 4 sites of Tau fibrils were shown that Site-1 was 25.4 kcal/mole, Site-2 was 38.0 kcal/mole, Site-3 was 33.7 kcal/mole, and Site-4 was 38.4 kcal/mole.

In this embodiment, the docking score of CDOCKER INTERACTION

ENERGY of the compound AI-INER-M-4 at 4 sites of Tau fibrils were shown that Site-1 was 35.4 kcal/mole, Site-2 was 51.5 kcal/mole, Site-3 was 43.6 kcal/mole, and Site-4 was 43.0 kcal/mole.

In this embodiment, the docking score of CDOCKER INTERACTION ENERGY of the compound AI-INER-M-5 at 4 sites of Tau fibrils were shown that Site-1 was 39.2 kcal/mole, Site-2 was 61.0 kcal/mole, Site-3 was 51.8 kcal/mole, and Site-4 was 46.7 kcal/mole.

In this embodiment, the docking score of CDOCKER INTERACTION

ENERGY of the compound AI-INER-M-6 at 4 sites of Tau fibrils were shown that Site-1 was 33.7 kcal/mole, Site-2 was 39.6 kcal/mole, Site-3 was 40.8 kcal/mole, and Site-4 was 39.3 kcal/mole.

In this embodiment, the combination of compound AI-INER-M-4, compound AI-INER-M-5, and compound AI-INER-M-6 all have higher docking score of

CDOCKER INTERACTION ENERGY at the four sites on Tau fibrils than those of the compound MK6240. The compound AI-INER-M-5 at the four binding sites of Tau fibrils all have the largest docking score of CDOCKER INTERACTION ENERGY in the experiment, which represents the binding of the compound AI-INER-M-5 in this experiment and Tau fibrils are the most stable.

In this embodiment, the docking scores of CDOCKER INTERACTION ENERGY of the compound AI-INER-M-1 at the four binding sites of Tau fibrils were similar to those of compound MK6240. The docking scores of CDOCKER INTERACTION ENERGY of the compound AI-INER-M-2 at Site-1, Site-2, and Site-3 of Tau fibrils were all similar to those of the compound MK6240. The docking score of CDOCKER INTERACTION ENERGY of the compound AI-INER-M-2 at Site-4 of Tau fibril was higher than that of MK6240. The docking scores of CDOCKER INTERACTION ENERGY of the compound AI-INER-M-2 at Site-1, Site-3 and Site-4 of Tau fibrils were all higher than thoses of the compound MK6240.

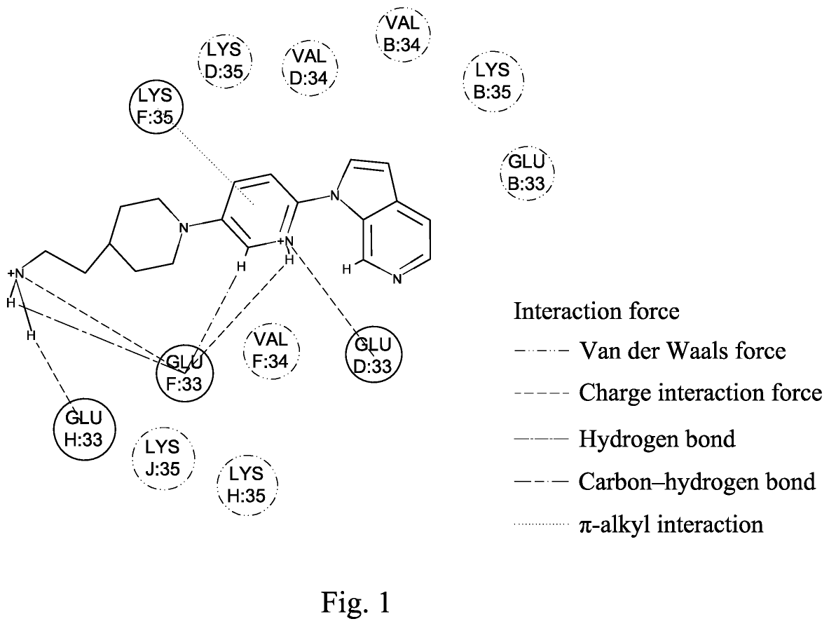

As shown in FIG. 1, it shows the force analysis of the compound AI-INER-M-5 and Site-2 of Tau fibril. The most important force is the pyridyl group of the compoundAI-INER-M-5 has charge interaction with glutamic acid 33 (GLU33) of the F chain and glutamic acid 33 of the D chain, as well as π-alkyl interaction lysine 35 (LYS35) of the F chain in Tau fibrils.

In summary, a series of compounds targeting Tau protein provided by the present disclosure will indeed bind to Tau protein. It is indeed a novel and progressive inventive step. A patent for invention was filed according to the law. The content is only a description of the preferred embodiments of the present disclosure. Any changes, modifications, alterations or equivalent substitutions extended according to the technical means and scope of the present disclosure should also fall within the scope of the patent application of the present disclosure.

What is claimed is:

1. A compound shown as formula (I), 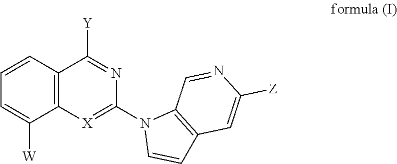
wherein
X is CH or N;
Y is I or H;
Z is NH₂ or H;
W is halogen or H.

2. A compound shown as formula (II), 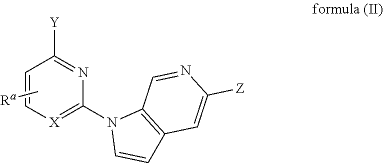
wherein
X is CH or N;
Y is I or H;
Z is NH₂ or H;
Ra is C₁₋₆ alkyl, C₂₋₆ alkenyl, C₂₋₆ alkynyl, C₁₋₆ haloalkyl, C₂₋₆ haloalkynyl, (CH₂)₁₋₆ pyrazolyl, C₁₋₆ borenyl, C₂₋₆ borenyl, or combinations of any one of the above-mentioned.

3. The compound according to claim 1, wherein the X position is CH, the Y position is H, the Z position is H, and the W position is F, and the structure of the compound is as follows:

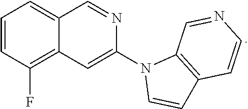 [SMILES: Fc1cccc2cnc(-n3ccc4ccncc43)cc12]

4. The compound according to claim 1, wherein the X position is CH, the Y position is H, the Z position is NH₂, and the \V position is H, and the structure of the compound is as follows:

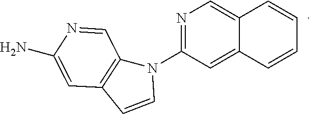 [SMILES: Nc1cc2ccn(-c3cc4ccccc4cn3)c2cn1]

5. The compound according to claim 2, wherein the X position is CH, the Y position is H, the Z position is H, and the Ra position is fluoromethyl and ethynyl, and the structure of the compound is as follows:

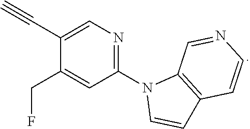 [SMILES: C#Cc1cnc(-n2ccc3ccncc32)cc1CF]

6. The compound according to claim 2, wherein the X position is N, the Y position is H, the Z position is H, and the Ra position is propylpyrazolyl, and the structure of the compound is as follows:

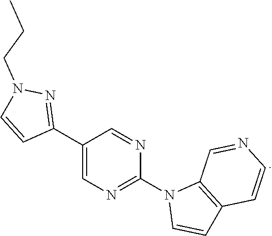 [SMILES: CCCn1ccc(-c2cnc(-n3ccc4ccncc43)nc2)n1]

7. The compound according to claim 2, wherein the X position is CH, the Y position is H, the Z position is H, and the Ra position is Piperidin-4-yl, and the structure of the compound is as follows:

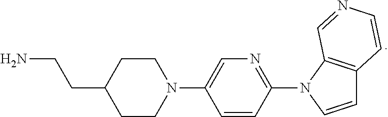 [SMILES: NCCC1CCN(c2ccc(-n3ccc4ccncc43)nc2)CC1]

8. The compound according to claim 2, wherein the X position is CH, the Y position is I, the Z position is H, and the Ra position is pyridyl, and the structure of the compound is as follows:

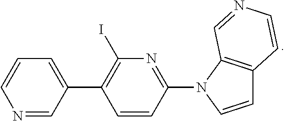 [SMILES: Ic1nc(-n2ccc3ccncc32)ccc1-c1cccnc1]

9. A method for preparing the compound according to claim 3, comprising steps as follows: 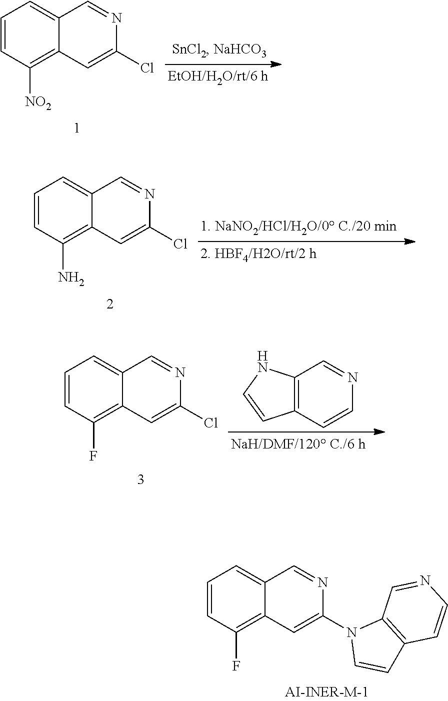
step 1:
dissolving compound 1 in ethanol and water, and adding stannous chloride and sodium bicarbonate wherein the number of mole of Compound 1 the number of mole of stannous chloride:the number of mole of sodium bicarbonate=1:2:2, and performing reaction at room temperature for 6 hours, and then performing concentration under reduced pressure and purification to obtain Compound 2;
step 2:
dissolving Compound 2 and sodium nitrite in 1N hydrochloric acid, performing reaction under nitrogen gas at 0° C. for 20 minutes, and adding fluoroboric acid then returning to room temperature and reacted for 2 hours, wherein the number of mole of Compound 2:the number of mole of sodium nitrite:the number of mole of fluoroboric acid=1:2:2, and then performing concentration under reduced pressure and purification to obtain Compound 3;
step 3:
adding Compound 3 with indole and sodium hydride to be dissolved in dimethylformamide and performing reaction at 120° C. for 6 hours, wherein the number of mole of Compound 3:the number of mole of indole:the number of mole of sodium hydride=1:1.5:1.5, and at last performing extraction and purification to obtain compound AI-INER-M-1.

10. A method for preparing the compound according to claim 4, comprising steps 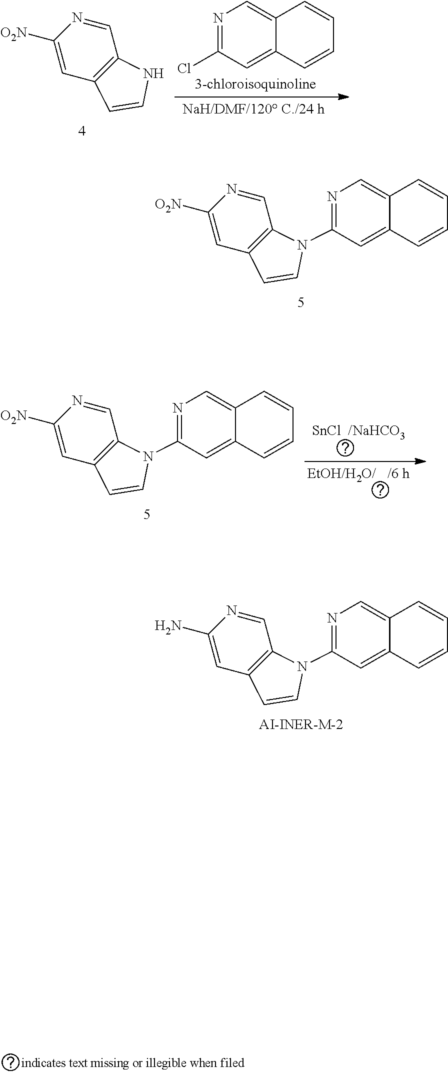
step 1:
dissolving Compound 4 in dimethylformamide, and adding 3-chloroiso-quinoline and sodium hydride to perform reaction at 120° C. for 24 hours, wherein the number of mole of Compound 4:the number of mole of 3-chloroisoquinoline:the number of mole of sodium hydride=1:1.5:1.5, and then performing extraction and purification to obtain Compound 5;
step 2:
dissolving Compound 5 in ethanol and double-distilled water, and adding stannous chloride and sodium bicarbonate to perform reaction for 6 hours at room temperature, wherein the number of mole of Compound 5:the number of mole of stannous chloride:the number of mole of sodium bicarbonate =1:1.5:1.5, and at last performing extraction and purification to obtain compound AI-INER-M-2.

11. A method for preparing the compound according to claim 5, comprising steps as follows: 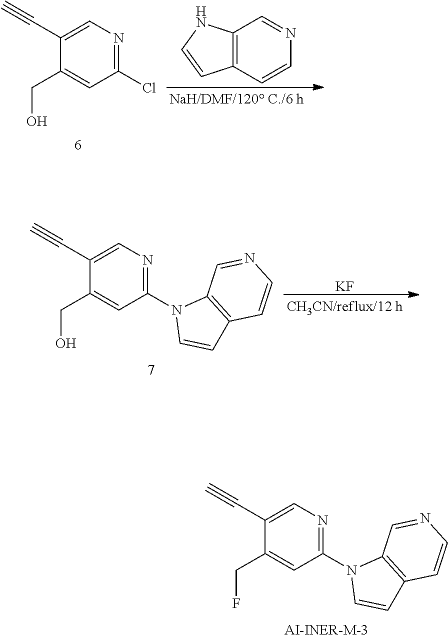 [SMILES: C#Cc1cnc(Cl)cc1CO ; c1cc2cc[nH]c2cn1 ; C#Cc1cnc(-n2ccc3ccncc32)cc1CO ; "KF" ; C#Cc1cnc(-n2ccc3ccncc32)cc1CF]
step 1:
dissolving Compound 6 in dimethylformamide and adding indole and sodium hydride to perform reaction at 120° C. for 6 hours, wherein the number of mole of Compound 6:the number of mole of indole:the number of mole of sodium hydride=1:1.5:1.5, and then performing extraction and purification to obtain Compound 7;
step 2:
dissolving Compound 7 and potassium fluoride in acetonitrile, and heating at reflux under a flow of nitrogen gas to perform reaction for 12 hours, wherein the number of mole of Compound 7:the number of mole of potassium fluoride=1:2, and at last performing concentration under reduced pressure and purification to obtain compound AI-INER-M-3.

12. A method for preparing the compound according to claim 6, comprising steps as follows: 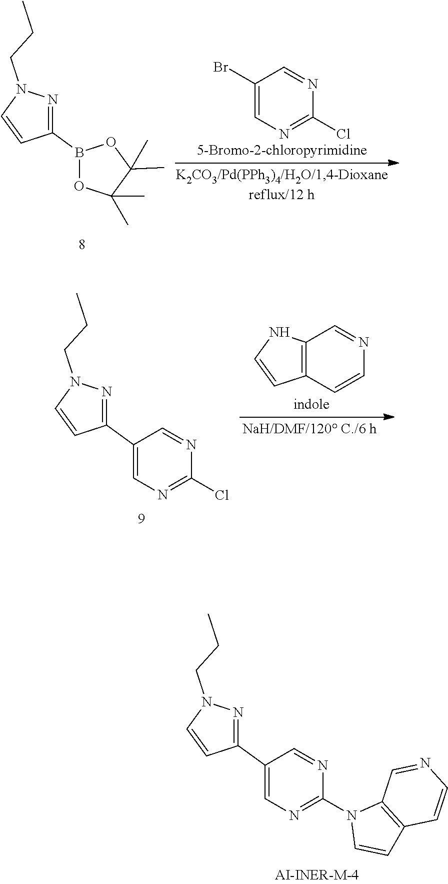 [SMILES: CCCn1ccc(B2OC(C)(C)C(C)(C)O2)n1 ; Clc1ncc(Br)cn1 ; CCCn1ccc(-c2cnc(Cl)nc2)n1 ; c1cc2cc[nH]c2cn1 ; CCCn1ccc(-c2cnc(-n3ccc4ccncc43)nc2)n1]
step 1:
dissolving Compound 8 and 5-bromo-2-chloropyrimidine in a mixture solution of water and 1,4-dioxane, and adding potassium carbonate and tetrakis(triphenylphosphine)palladium, heating at reflux to perform reaction for 12 hours, wherein the number of mole of Compound 8:the number of mole of 5-bromo-2-chloropyrimidine: the number of mole of potassium carbonate:the number of mole of tetrakis(triphenylphosphine)palladium=1:1:2:0.05, and then performing concentration under reduced pressure and purification to obtain Compound 9;
step 2:
adding Compound 9 with indole and sodium hydride to be dissolved in dimethylformamide and reacted at 120° C. for 6 hours, wherein the number of mole of Compound 9:the number of mole of indole:the number of mole of sodium hydride=1:1.5:1.5, and then performing concentration under reduced pressure and purification, and at last obtaining compound AI-INER-M-4.

13. A method for preparing the compound according to claim 7, comprising steps 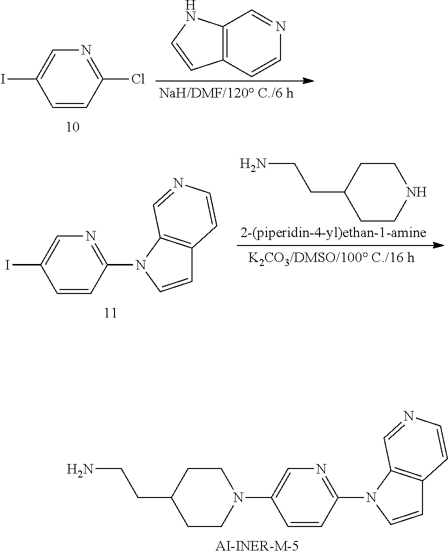 [SMILES: Clc1ccc(I)cn1 ; c1cc2cc[nH]c2cn1 ; Ic1ccc(-n2ccc3ccncc32)nc1 ; NCCC1CCNCC1 ; NCCC1CCN(c2ccc(-n3ccc4ccncc43)nc2)CC1]
step 1:
dissolving Compound 10 in dimethylformamide, adding indole and sodium hydride to perform reaction at 120° C. for 6 hours, wherein the number of mole of Compound 10:the number of mole of indole:the number of mole of sodium hydride=1:1.5:1.5, and then performing extraction and purification to obtain Compound 11;
step 2:
dissolving Compound 11 in dimethyl sulfoxide and adding 2-(piperidin-4-yl) ethan-1-amine and potassium carbonate, heating to a temperature of 100° C. to perform reaction for 16 hours, wherein the number of mole of Compound 11:the number of mole of 2-(piperidin-4-yl)ethan-1-amine:the number of mole of potassium carbonate=1:1.5:1.5, and at last performing concentration under reduced pressure and purification to obtain compound AI-INER-M-5.

14. A method for preparing the compound according to claim 8, comprising steps as follows: 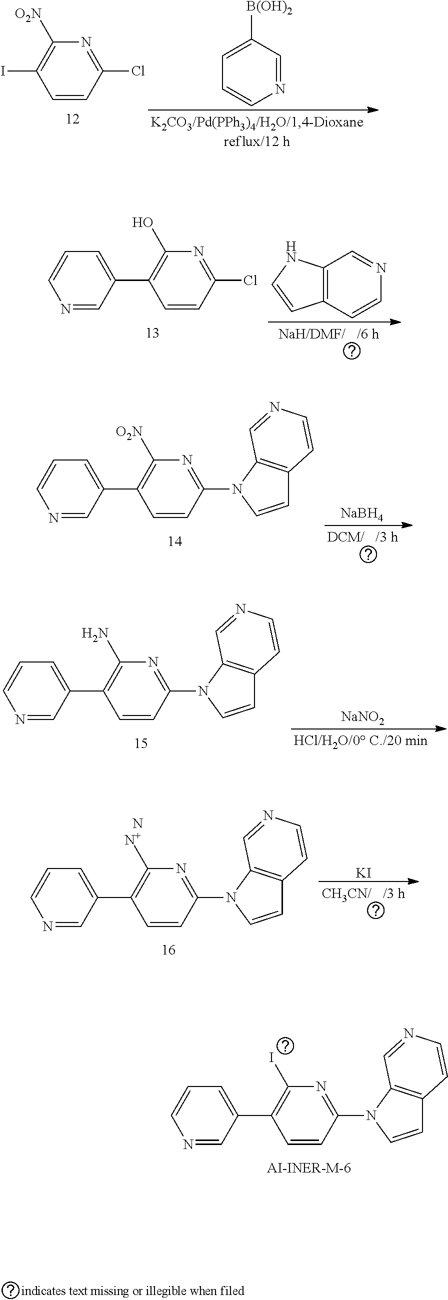
step 1:
dissolving Compound 12 in a mixture solution of water and 1,4-dioxane, and adding 3-pyridinylboronic acid, potassium carbonate and tetrakis(triphenylphosphine)palladium, heating at reflux to perform reaction for 12 hours, wherein the number of mole of Compound 12:the number of mole of 3-pyridinylboronic acid:the number of mole of potassium carbonate:the number of mole of tetrakis(triphenylphosphine)palladium=1:1:2:0.05, and then performing extraction and purification to obtain compound 13;
step 2:
dissolving Compound 13 in dimethylformamide and adding indole and sodium hydride to perform reaction at room temperature for 6 hours, wherein the number of mole of Compound 13:the number of mole of indole:the number of mole of sodium hydride=1:1.5:1.5, and then performing extraction and purification, and at last obtaining compound 14;
step 3:
dissolving Compound 14 in dichloromethane and adding sodium borohydride to react at room temperature for 3 hours, wherein the number of mole of compound 14:the number of mole of sodium borohydride=1:1.5, and at last performing extraction and purification to obtain compound 15;
step 4:
dissolving compound 15 in 1N hydrochloric acid and adding sodium nitrite, performing reaction of the mixture under nitrogen gas at 0° C. for 20 minutes, wherein the number of mole of Compound 15:the number of mole of sodium nitrite=1:1.5, and obtaining Compound 16; draining Compound 16 and directly proceeding the next reaction, adding potassium iodide and acetonitrile then performing reaction at room temperature for 3 hours, wherein the number of mole of compound 16:the molar number of potassium iodide=1:2, and the iodine in potassium iodide is selected from the group consisting of ¹²³I, ¹²⁴I, ¹²⁷I and ¹³¹I, and at last performing concentration under reduced pressure and purification to obtain compound AI-INER-M-6.

15. A use of the compound according to claim 1 for the imaging of Tau protein.

16. A use of the compound according to claim 2 for the imaging of Tau protein.
